(19) United States
(12) Patent Application Publication
(10) Pub. No.: US 2025/0032444 A1
(43) Pub. Date: Jan. 30, 2025

(54) Prevention and treatment of depressive disorders and conditions promoted by protease containing plasma extracellular vesicles (PCpEV)

(71) Applicant: Evexys Biotech Oy, Espoo (FI)

(72) Inventors: Andreas Baur, Erlangen (FI); Erkko Ylösmäki, Espoo (FI)

(21) Appl. No.: 18/714,605

(22) PCT Filed: Nov. 30, 2022
(86) PCT No.: PCT/FI2022/050801

§ 371 Date: May 30, 2024

(30) Foreign Application Priority Data
Nov. 30, 2021 (FI) 20216228
Jul. 7, 2022 (FI) 20225643

(51) Int. Cl. A61K 31/352; A61K 35/00; G01N 33/68

(52) U.S. Cl. CPC A61K 31/352; A61K 35/00; G01N 33/6896; G01N 2333/91102; G01N 2500/10; G01N 2800/52

(57) ABSTRACT

The present invention is directed to 4-methylumbelliferone (hymecromone) or a derivative thereof for use in the treatment of major depressive disorder, mood disorders, anxiety-related disorders and depression associated with diseases or drug treatments including Alzheimer's disease, HIV associated neurocognitive disorders (HAND), psoriasis, chronic fatigue syndrome, Parkinson's disease, Long COVID syndrome and drug treatment regimens with IFN-alpha or vitamin A analogues, promoted by pathogenic extracellular vesicles, wherein 4-methylumbelliferone inhibits hyaluronic acid (HA) synthases and block the incorporation of HA and/or low molecular weight cleavage products into said extracellular vesicles. The present invention is also directed to a method for monitoring the efficacy of the treatment and delivery of therapeutic cargo to cells incorporating pEV by means of HA-binding receptors. The present invention further provides an in vitro screening method for identifying further inhibitors of HA synthases.

### FIELD OF THE INVENTION

The present invention is based on the discovery that the liver reacts to the development of diseases, in part through a release of large amounts of protease-containing plasma extracellular vesicles (PCpEV) that enter target cells, as for example in the choroid plexus (CP) using Hyaluronan (HA) as a ligand. The present invention is directed to inhibit hyaluronic acid synthase (HAS) by 4-methylumbelliferone (4-MU) and derivatives thereof in order to prevent the release of PCpEV containing HA that can enter HA-receptor containing cells, like the CP, therefore delaying the onset of depression and symptoms associated with the inflammation in the brain, commonly referred to as brain fog. Furthermore, extracellular vesicles bearing Hyaluronan on their surface can be designed to transfer effector molecules into the brain (designer vesicles), for example for reversing disease conditions or aging.

### BACKGROUND OF THE INVENTION

Major depressive disorder (MDD) is a frequent and severe psychiatric condition, having an estimated prevalence of around 15% in the general population. It is twice as prevalent in women compared to men. One of the reasons for MDD seems to be reduced neurotransmission of brain monoamines. Hence, drugs that enhance neurotransmission may be used to treat MDD. However, only one third of patients suffering from depression achieve complete remission of symptoms after a single antidepressant treatment. Therefore, other underlying pattern mechanisms exist. Numerous studies have described a correlation between depression and peripheral markers of inflammation in blood and cerebrospinal fluid. Supporting this assumption, pro-inflammatory agents, as for example IFN alpha, can induce MDD as a side effect, whereas inhibitors of inflammation, like non-steroidal anti-inflammatory drugs (NSAIDS) show antidepressant effects.

After the acute phase of Coronavirus Disease 2019 (COVID-19) caused by SARS-CoV-2 infection persistent and prolonged symptoms can be observed in several patients irrespective of the severity of their primary disease. The pathogenesis of this Post-Acute COVID-19 Syndrome or Long-COVID is not well understood, but involves chronic inflammation and immune activation. The primary symptoms of Long-COVID include depression, anxiety, fatigue, and cognitive impairments that can collectively be described a brain fog. No established therapy for long-COVID currently exists, but different antidepressant drugs are commonly used and various anti-inflammatory therapies are under investigation.

By analyzing the abundance/number, content, function and origin of PCpEV in patients with neurologic and neuropsychiatric disorder, including depression, we concluded that PCpEV are highly inflammatory, derive from the liver, are constantly secreted in very high numbers, up to 100-fold higher than those observed in healthy controls. As determined in animal experiments, PCpEV have a striking tropism for the choroid plexus (CP). Immunohistochemistry of brain tissue from mice treated with a single injection of PCpEV revealed that from the CP these vesicles reach paraventricular neurons in the thalamus and the limbic system, both of which are necessary to give rise to new neurons in adults. Based on recent findings, this hippocampal neurogenesis may be crucial in the pathophysiology of depression as well as in the effect of antidepressant drugs. In vitro experiments using a neuronal cell line demonstrated that PCpEV induced non-physiological events in target cells including cleavage of proTNF and the amyloid precursor protein APP. Taken together, these findings suggested that PCpEV may be involved in the pathogenesis of MDD.

Further experiments revealed that the affinity of PCpEV for cells of the plexus choroideus is mediated by hyaluronic acid (HA) and its interaction with HARE/Stabilin-2. The HA polymer is produced by three isotypes of the hyaluronic acid synthase (HAS-1, -2 and -3) yielding a high molecular weight glucosaminoglycan. The polymer is produced by many cells in the human body. It is not only part of the extracellular matrix (ECM) but also has many roles in normal tissue function and development, providing support in cell anchorage, facilitating cell to cell signaling as well as cell movement and migration. HA is processed by hyaluronidases into a continuum of different sized HA polymers, including low molecular weight HA (LMW-HA; <120 kDa). Whereas high molecular weight HA polymers (HMW-HA; >400 kDa) predominate in most healthy tissues and have anti-inflammatory effects, LMW-HA polymers predominate it sites of active inflammation. We found that the LMW-HA is also found in abundance on PCpEV from patients with depression.

The three isotypes of HAS are efficiently inhibited by 4-methylumbelliferone also called Hymecromone. There are at least two molecular mechanisms describing this inhibitory effect: first, Hymecromone functions as a competitive substrate for an enzyme (UDP-glucuronosyltransferase) involved in HA synthesis. Second, Hymecromone binds covalently to glucuronic acid. As a consequence the concentration of the HA precursor UDP-GlcUA is reduced. In addition, Hymecromone reduces the expression of HAS mRNA by a mechanism that is not well understood. Extracellular vesicles produced in the presence of Hymecromone lack HA and additional components of PCpEV. Furthermore, PCpEV produced under these conditions lose their affinity for the CP.

Hymecromone is an already established drug for humans, originally used for its anti-spasmodic activity. In Europe, the drug is approved for biliary dyskinesia, for example under the generic name Cantabilin in Italy. The typical approved dosing for adults is 300 to 800 mg given orally three times per day. At least 182 patients have been treated in clinical trials and no serious side effects were reported. The longest reported duration of administration was 1200 mg per day for three months. The most common side effects during treatment was mild diarrhea in 1 to 10% of patients. Hymecromone is efficiently metabolized predominantly by the liver through conjugation of glucuronic acid and eliminated in the bile and urine. As a result of this efficient metabolism, the fraction of unchanged Hymecromone reaching systemic circulation is low. However, the high extraction by the liver may be beneficial as the drug concentrates in the hepatic system where PCpEV are produced.

MDD disease is diagnosed predominantly by clinical criteria. In a new development we have determined that PCpEV contain factors that reliably allow the diagnosis of MDD by biomarker pattern and a protease activity assay. These test systems may be used to verify the effect of Hymecromone.

### SUMMARY OF THE INVENTION

We have discovered that the pathogenesis of depression involves secretion of highly inflammatory and protease-containing plasma extracellular vesicles (PCpEV). Although the contents of these pathogenic vesicles vary from one disease to another, a panel of key components such as ADAM10, ADAM17, Hck, and certain matrix metalloproteinases (MMP) are typically present, and can be found in PCpEV isolated from patients with diseases as diverse as HIV infection, cancer, and MDD, but notably not from healthy controls. It is known that hyaluronic acid (HA) and HAS synthetases (HAS1-3) are somehow involved in extracellular vesicle biogenesis. HA is the natural ligand for CD44, and an important component of normal extracellular matrix. Furthermore, it has been reported that cleavage products of HA may be pro-inflammatory, and involved in the pathogenesis of various diseases. The present invention is based on our observation that HA and HA cleavage products (LMW-HA) are found on PCpEV in MDD. Strikingly, we found that pharmacological inhibition of HAS activity with 4-Methylumbelliferone (Hymecromone) efficiently blocked not only the incorporation of LMW-HA into extracellular vesicles, but also the incorporation of ADAM10/17, Hck, and MMPs. Furthermore, such pEV failed to enter the brain through the CP as demonstrated in animal injection experiments. Thus, Hymecromone and other HAS inhibitors constitute a promising new therapeutic modality for treating MDD and other diseases promoted by PCpEV. In addition to reducing the inflammatory nature of PCpEV, Hymecromone therapy is beneficial by interfering with HA-dependent entry of PCpEV into the brain via the CP. Measuring PCpEV associated LMW-HA, ADAM10/17, Hck, and MMPs provide diagnostic means for assessing the indication for, as well as monitoring of such therapy. Hymecromone is already approved for the treatment of humans for disorders of the bile ducts. Via drug repurposing it could be an effective medication for MDD and depression associated with diseases.

Accordingly, the present invention is directed to a compound having the structure of Formula

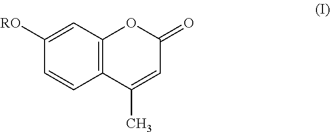

wherein R is H, a phosphate, ester, monosaccharide, or disaccharide;
or a salt thereof,
for use in the treatment and/or prevention of a condition selected from the group consisting of major depressive disorder, mood disorders, anxiety-related disorders and depression associated with diseases or drug treatments including Alzheimer's disease, HIV associated neurocognitive disorders (HAND), psoriasis, chronic fatigue syndrome, Parkinson's disease. Long COVID syndrome and drug treatment regimens with IFN-alpha or vitamin A analogues.

In another aspect, the invention provides a pharmaceutical composition comprising the compound as defined above and at least one of the following: a physiologically acceptable carrier, buffer, excipient, preservative and stabilizer for use in the treatment of a condition selected from the group consisting of: major depressive disorder, mood disorders, anxiety-related disorders and depression associated with diseases or drug treatments including Alzheimer's disease, HIV associated neurocognitive disorders (HAND), psoriasis, chronic fatigue syndrome, Parkinson's disease, Long COVID syndrome and drug treatment regimens with IFN-alpha or vitamin A analogues.

In another aspect, the invention provides an in vitro screening method for identifying inhibitors of HA synthases, comprising: a) incubating a liver cell expressing a HA synthase in the presence of a candidate compound, b) comparing the level of low molecular weight HA in extracellular vesicles released from the cell in the presence of the candidate compound to the level of low molecular weight HA in extracellular vesicle released from the cell in the absence of the candidate compound; c) comparing the activity and/or expression level of the HA synthase in the presence of the candidate compound to the activity and/or expression level of the HA synthase in the absence of the candidate compound; d) selecting those candidate compounds which inhibit the HA synthase and block the incorporation of low molecular weight HA into extracellular vesicles.

In another aspect, the invention provides a method for monitoring the efficacy of a treatment with an inhibitor of a HA synthase, preferably hymecromone, i.e. 4-methylumbelliferone, or a salt thereof, in a patient, the method comprising the steps of providing a blood sample taken from the patient subjected to said treatment, purifying extracellular vesicles (EV) from said sample and detecting the presence or amount of low molecular weight HA in said vesicles, wherein a condition treated with said inhibitor of a HA synthase is preferably selected from the group consisting of: major depressive disorder, mood disorders, anxiety-related disorders and depression associated with diseases or drug treatments including Alzheimer's disease, HIV associated neurocognitive disorders (HAND), psoriasis, chronic fatigue syndrome, Parkinson's disease, Long COVID syndrome and drug treatment regimens with IFN-alpha or vitamin A analogues as well as other diseases associated with innate inflammatory conditions.

In another aspect, the invention provides method for transferring therapeutic cargo to the brain via the choroid plexus by means of vesicular structures containing HA on the surface in order to treat or reverse disease conditions including: major depressive disorder, mood disorders, anxiety-related disorders and depression associated with diseases or drug treatments including Alzheimer's disease, HIV associated neurocognitive disorders (HAND), psoriasis, chronic fatigue syndrome, Parkinson's disease, Long COVID Syndrome and drug treatment regimens with IFN-alpha or vitamin A analogues, wherein said treatment comprises a step of administering in vitro generated pEV containing HA and/or derivatives thereof, e.g. low molecular weight HA, along with therapeutic cargo, for example enzymes, non-enzymatic effectors, mRNA, micro-RNA, peptides, antisense RNA, and DNA.

Such vesicles might be generated by overexpression of a HAS expression plasmid (e.g. HAS1, HAS2 or HAS3), for example in a cell line, Ike for example derived from immortalized hepatocytes, or a primary cell, Ike for example dendritic cells or primary hepatocytes. These cells could be incubated, as well as transfected, in GMP-compliant commercial cell manufacturing devices (e.g. from Miltenyi Biotech), from which cell supernatants can be regularly obtained and purified for extracellular vesicles by various methods, for example by size exclusion chromatography that avoids the contamination of purification proteins, as for example antibodies. Along with the HAS expression plasmid, the specific therapeutic cargo may be transfected into EV producing cells, or, alternatively, stably expressed in these EV-producing cells. This could, for example, comprise factors Ike proteins, cytokines, chemokines, mRNA, micro-RNA, kinases, phosphatase and proteases. The HA surface content may be assessed by FACS analysis using an commercially available HA-binding protein assay and/or free flow electrophoresis (FFE), that allows discrimination and quantification of vesicles based on their surface charge. The latter changes greatly with the presence of HA (negative charge). The specific content of the vesicles could be verified and monitored by various methods, including PCR, Western blot, FACS analysis and enzymatic assays.

### BRIEF DESCRIPTION OF THE DRAWINGS

FIG. 1: Plasma extracellular vesicles (pEV) from a patient with depression associated with Alzheimer's Disease contain different size fractions of hyaluronic acid (HA) and Hymecromone inhibits hyaluronic acid uptake into EV.

(A): Western blot of gradient purified pEV from one representative depression patient and a healthy control. The arrows indicate the various size fractions of HA. CD81 served as a loading control for pEV. Cell lysates from 293 T cells served as positive control for HA.

(B): Hymecromone inhibits the uptake of HA into EV. Huh7 liver cells were treated to induce expression and activity of HAS similar as seen in a depression patient with AD (panel A). Subsequently EV were purified from culture supernatant of Huh7 cells that were not treated for HAS induction (nil), from Huh7 cells that were treated for HAS induction (48 h; HAS-nd.) and from Huh7 cells that were treated for has induction and additionally incubated with Hymecromone (HAS-ind.+HyCr.). CD81 served as a loading control for pEV. Cell lysates from 293 T cells served as positive control for HA.

FIG. 2: Hymecromone inhibits uptake of ADAM10 and 17 and Hck into EV.

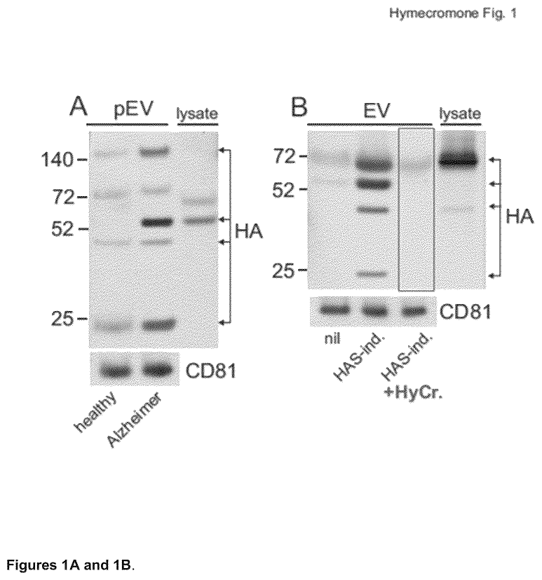

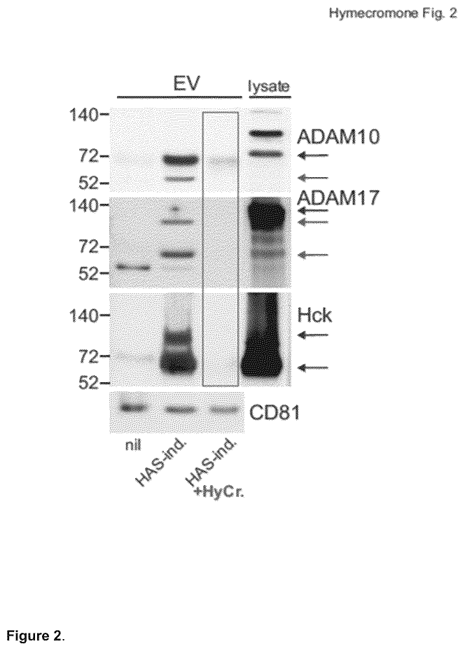

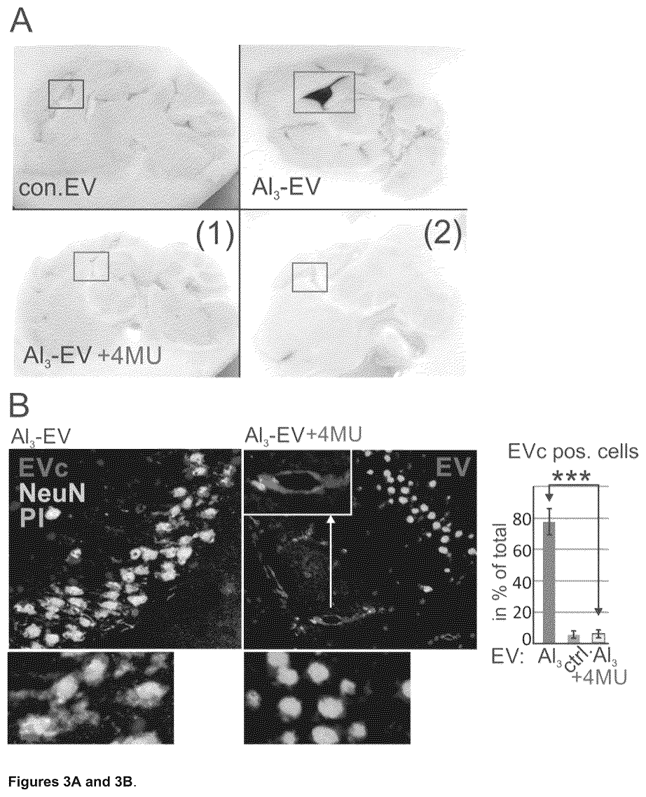

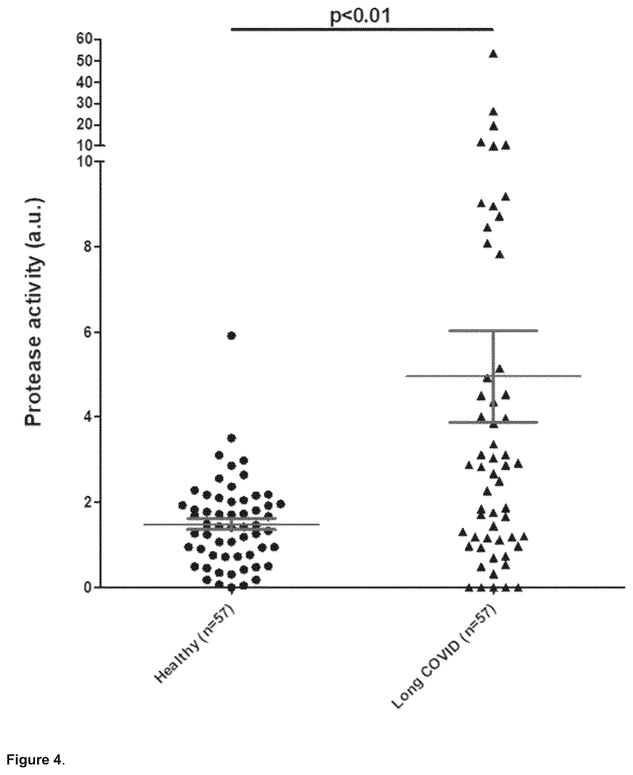

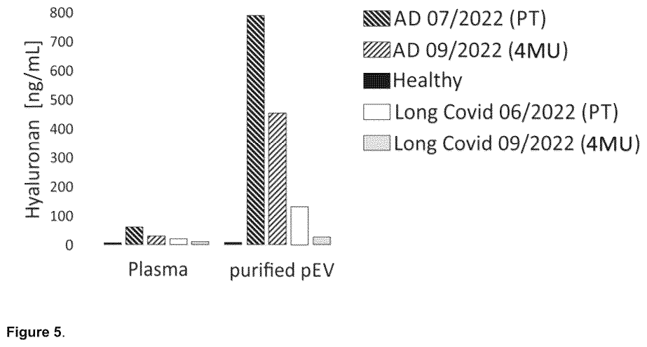

Huh7 liver cells were treated to induce expression and activity of HAS similar as seen in a depression patient (FIG. 1A). Subsequently EV were purified from culture supernatant of Huh7 cells that were not treated for HAS induction (nil), from Huh7 cells that were treated for HAS induction (48 h; HAS-ind.) and from Huh7 cells that were treated for HAS induction and additionally incubated with Hymecromone (HAS-nd.+HyCr.). Red arrows indicate the enzymatic active form of ADAM10 and ADAM17. CD81 served as a loading control for pEV. Cell lysates from 293 T cells served as positive control for HA.

FIG. 3: Hymecromone/4-methylumbelliferone (4MU) blocks EV upload and CP targeting.

(A) Accumulation/absence of accumulation of labeled pEV (red) in the Choriod Plexus 72 h after injection of 4MU-treated EV into tail veins of a mouse (n=4/condition). (B) IHC of a hippocampal section and quantification of EVc pos. cells. Statistical significance by one-way ANOVA and Tukey's test, ***p<0.005.

FIG. 4: Patients with Long Covid have on average a higher protease activity in pEV as compared to healthy individuals. Plasma EV from 57 health individuals and 57 patients with clinically confirmed Long Covid syndrome were analyzed for protease activity. For this assay, vesicles were purified from 2 milliliter of plasma by dual-mode chromatography (DMC). Subsequently aliquots of the isolated pEV were incubated with 15 different FRET peptides, each specific for a single protease. The average activity of al proteases for one individual were then plotted on a graph. The difference between health individuals and patients with Long COVID was significantly different (Student's t-test).

FIG. 5: Treatment of patients with 4MU. A 55 year old female patients with a confirmed diagnosis of Alzheimer's disease (AD)-associated depression (improved significantly under treatment) and a 32 old patient with confirmed Long COVID were treated with 4×300 mg 4MU beginning in July 2022 and June 2022 (fully recovered under treatment), respectively. Samples were taken before treatment (PT) and after 2.5 and 3 months of treatment (4MU) and analyzed for the presence of HA in plasma and on purified pEV (determined by HA-ELISA) before and during 4MU treatment.

### DETAILED DESCRIPTION OF THE INVENTION

PCpEV derived from liver cells (hepatocytes) reach the brain by interacting with a receptor in cells of the CP. For this interaction they contain HA. Hymecromone not only blocks the uptake of HA into PCpEV but also alters the composition and reduces the inflammatory potential of these vesicles.

Hymecromone is approved for treatment of humans in the dosage of up to 2400 mg orally per day without causing serious side effects. Treatment of humans with the intention to block the pathology of MDD requires proper clinical diagnosis and ideally also assessment of biomarkers directly related to the effects of Hymecromone. These biomarkers are assessed in PCpEV after isolation from plasma.

Before humans are treated with Hymecromone, the presence of PCpEV can be confirmed using diagnostic procedures described below. In addition, it is preferable to verify the reduction of protease activity, HA and/or other inflammatory content in PCpEV by treatment with Hymecromone. These markers are found in abundance in PCpEV from patients with MDD but are reduced or abolished upon successful treatment with Hymecromone. The assessment of these markers is an indicator for successful treatment. The diagnosis and therapy described here represents a significant advancement in the treatment of MDD.

### Procedure of PCpEV/pEV Isolation for the Assessment of Biomarkers

Various aspects and embodiments of the present invention will now be described in more detail by way of example, with particular reference to the separation of pEV using techniques of differential centrifugation and immunoseparation. It will be appreciated that modification of detail may be made without departing from the scope of the invention.

In order to measure biomarkers in pEV, they have to be separated from other plasma components and concentrated into a small volume. Any method allowing concentration and separation of pEV is both suitable and required to assess pEV biomarkers. Suitable methods are for example: differential ultracentrifugation, ultracentrifugation in combination with gradient fractionation, antibody coupled matrices (e.g. beads or filters), exchange/spin-column based methods and pEV-binding resins.

Preferably, the pEV are separated from the plasma of an individual or experimental animal model. Techniques for the separation of plasma from blood will be clear to the skilled reader. However, suitable fluids include bodily fluids such as blood, ascites and urine and growth medium in which cells are cultured in vitro. The individual from which blood plasma is taken for preparation of pEV may be any animal. Suitable animals include primates, preferably higher primates such as chimpanzees. Most preferably, pEV are prepared from human patients.

It has been discovered that the purification of pEV from 50 μl-15 ml plasma allows the preparation of sufficient quantities of biomarkers for assessment. This method/procedure is firstly described in a paper describing the biomarker content of HIV pEV in plasma (Lee et al., 2016) showing the content of 25 specific markers. Preferably, pEV are prepared by differential centrifugation, according to the technique of Raposo et al. (Raposo et al., 1996).

According to one embodiment of the invention, the assessment of pEV-derived biomarkers comprises the sequential steps of centrifuging plasma obtained from an individual to give a pellet that is enriched in pEV, and isolating said biomarkers from said pEV. For pEV purification patient plasma samples are diluted 1:1 with PBS and centrifuged for 30 min at 2,000 g, 45 min at 12,000 g and ultra-centrifuged for 2 h at 110,000 g. Pellets are resuspended in 10 ml PBS and centrifuged at 110,000 g for 1 h. Pellets are finally resuspended in 100 μl PBS and considered as EV preparations.

As an alternative purification method, plasma samples may be incubated with beads coated with antibody that recognises marker molecules on the surface of pEV. For example, anti-αvβ3 or anti-ADAM10 antibodies may be used in this respect. As the skilled reader will appreciate, magnetic beads, such as those manufactured by Dynabeads, Dynal, Oslo, Norway, or polystyrene beads (for example, those made by Pierce) are particularly suitable in this embodiment of the invention. Other alternatives for the purification of pEV include the use of sucrose density gradients or organelle electrophoresis (Tulp et al., 1994).

### Biomarker Assessment

The primary method to determine the protease content (enzymatic biomarkers) is using a patented system assessing their physiological activity by means of FRET peptides. This requires a certain purity of pEV, as for example achieved by Dual Mode Chromatography (DMC), a combination of size exclusion chromatography and retention/exclusion of positively charged non-specific complexes (e.g. lipoproteins) by a cation exchange matrix, by antibody- or equivalent affinity-based isolation, or by sucrose gradient.

HA may be quantified in pEV using a colorimetric assay using Hyaluronidase and chitinases, both of which are commercially available.

In order to measure and quantify the non-enzymatic biomarker content, purified pEV are resuspended in a lysis buffer. Preferably this is done in a small volume adjusted to the requirements of the readout assay (for example 10 μl for factor assessment by the Olink technology, or semi-quantitative Western blot analysis by Ray Biotech, or antibody coated beads for FACS analysis (e.g. from BioLegend), or for miRNA quantification by the Nanostring technology microRNA microarray by Agilent).

One technique that is suitable for analyzing pEV protein/peptide content is by SDS-PAGE and Western blotting, using antibodies directed against proteins/peptides that are contained in PCpEV. Antibodies directed against ADAM10, ADAM17, all matrix metalloproteinases, HA, Chitinases, GRASP55, PDL-1, FasL and Hck components are particularly suitable. Binding of these primary antibodies to pEV can be assessed using, for example, labelled secondary antibodies that bind to the primary antibodies. For example, anti-ADAM10 monoclonal antibody can be used as the primary antibody, whilst a labelled anti-mouse IgG can be used as the secondary antibody.

Alternatively, any assay system that identifies/recognizes individual proteins or peptides in a concentrated pEV preparation is suitable to assess the biomarker composition. Ideally, this assessment is performed in a quantitative or semi-quantitative manner to judge the relative magnitude/concentration of a signal/biomarker. Conventional methods to recognize these components are a combination of specific antibodies and complementary DNS tags (Olink technology), or on a filter surface (e.g. protein array from Ray Biotech) or a plastic bead and assessed by FACS analysis (e.g. multiplex array from Biolegend).

The following CCF and MP factors and combinations thereof are particularly suitable for the diagnosis and monitoring of acute and chronic diseases: ADAM10, ADAM17, all matrix metalloproteinases, HA, Chitinases, GRASP55, PDL-1, FasL and Hck.

The markers listed above characterize the presence of a specific class of PCpEV in comparison to EVs from healthy individuals. The absence of HA would indicate that the patient would be unlikely to benefit from treatment with Hypercromone.

Any technology able to assess micro-RNAs in a quantitative or at least semi-quantitative way is suitable for the assessment of the pEV micro-RNA content in pEV. Particularly suitable is the assessment and quantification by micro-RNA microchip as commercially offered by Agilent, and the assessment by the novel NanoString technology. Alternatively, micro-RNAs may be quantified by quantitative PCR.

Micro-RNAs from pEV may be obtained and prepared for quantification by miRNA microchips as follows and described in Lee and colleagues (2016): pEV are purified and pelleted by differential ultracentrifugation as described above (alternatively by DMC). The pEV pellets are then dissolved in 700 μl of Qiazol and total RNA is isolated for example by using Qiagen miRNeasy Mini Kits (Qiagen 217004) according to the manufacturer's instructions. 100 ng of the extracted RNA is concentrated to 50 ng/μL and Cy3-labelled using Agilent's miRNA Complete Labeling and Hyb Kit (Agilent Technologies, 5190-0456). After purification through Micro Bio Spin Columns (Bio Red, 732-6221) the total RNA samples are hybridized for 20 hours at 55° C. to human miRNA microarrays (e.g. Agilent, Version V16, 8×60K). The microarrays are washed in Triton-containing washing buffer as recommended by the manufacturer and scanned with the Agilent's Microarray Scanner System (Agilent Technologies). The image files are analyzed and processed by Agilent Feature Extraction Software (Version 10.7.3.1). Increase of selected miRNAs in patients is determined by relative fold increase over healthy controls (microarray, quantitative PCR)) or by determining absolute copy numbers in comparison to healthy controls (Nanostring). The latter may be done by comparison with data stored in a data bank once increasing numbers of samples are analyzed.

Treatment with Hymecromone

In vitro a concentration of 300 μmol was sufficient to completely block the uploading of HA, Hck, and ADAM10 into vesicles. Whether these concentrations can be reached in circulation after oral uptake is not clear, as Hymecromone is efficiently metabolized in the Ever. On the other hand PCpEV are generated by liver cells where Hymecromone is metabolized. Hence, the oral dosage necessary to inhibit PCpEV in vivo cannot be extrapolated at present. Furthermore, there are likely inter-individual differences with respect to Hymecromone metabolization. Hence, the optimal dosage for each patient has to be determined by monitoring and analyzing the content of HA in PCpEV after therapy was started.

Hymecromone is usually given 4 to 6 times 300 to 400 mg or 1200 to 2400 mg per day. One example to treat patients would be to start a regimen with 3×300 mg per day and increase the dosage gradually until HA uptake into PCpEV is completely blocked (for analysis of PCpEV from plasma samples see above). Chemical Modifications of the drug may produce compounds that are less efficiently metabolized. Hymecromone may also be given intravenously or injected into the muscle or applied through an epidermal patch.

### Experimental Section

Plasma Extracellular Vesicle Purification (pEV):

Plasma EV purification was performed as previously described (Lee at al., 2016). Briefly, 12 ml blood plasma was diluted with 12 ml PBS and centrifuged for 30 min at 2000 g, 45 min at 12000 g and ultra-centrifuged for 2 h at 110,000 g. Pellets were washed in 32 ml PBS and pEV were ultra-centrifuged for 1 h at 110,000 g. Pellets were resuspended in a final volume of 120 μl, resulting in an equivalent of 1 ml plasma in 10 μl pEV-suspension.

### Transient Transfections and Immunoblotting:

293Tcells were cultured in DMEM (Lonza), 10% (v/v) fetal bovine serum (FBS) with Penicillin-Strepto-mycin at 37° C., 5% CO₂. For transient transfection, plasmids encoding the indicated proteins were transfected with Lipofectamine® LTX with Plus™ Reagent (Invitrogen) according to the manufacturer's instructions, or using the classical calcium phosphate procedure. Cells were used for experiments 24-72 h after transfection. Hymecromone was added at a final concentration of 300 mmol after transfection. For immunoblotting 20 μg of cellular protein lysate and 10 μg of EV lysate were loaded per lane. The latter corresponded to the production/secretion of 2-4 mio 293T cells within 48 h transfected and grown in one 10 cm dish.

### REFERENCES

Lee, J. H., Schierer, S., Blume, K., Dindorf, J., Wittki, S., Xiang, W., Ostalecki, C., Koliha, N., Wild, S., Schuler, G., Fackler, O. T., Saksela, K., Harrer, T., and Baur, A. S. (2016). HIV-Nef and ADAM17-Containing Plasma Extracellular Vesicles Induce and Correlate with Immune Pathogenesis in Chronic HIV Infection. EBioMedicine. 6, 103-113.
Raposo, G., Nijman, H. W., Stoorvogel, W., Liejendekker, R., Harding, C. V., Melief, C. J., and Geuze, H. J. (1996). B lymphocytes secrete antigen-presenting vesicles. J. Exp. Med. 183, 1161-1172.
Tulp, A., Verwoerd, D., Dobberstein, B., Ploegh, H. L., and Pieters, J. (1994). Isolation and characterization of the intracellular MHC class 11 compartment. Nature 369, 120-126.

1.-18. (canceled)

19. A method for monitoring the efficacy of a treatment with an inhibitor of a HA synthase in a patient, the method comprising the steps of:
providing a blood sample taken from the patient subjected to said treatment,
purifying extracellular vesicles (EV) from said sample and detecting the presence or amount of low molecular weight HA in said vesicles,
wherein a condition treated with said inhibitor of a HA synthase is selected from the group consisting of: major depressive disorder, mood disorders, anxiety-related disorders and depression associated with diseases or drug treatments, HIV associated neurocognitive disorders (HAND), psoriasis, chronic fatigue syndrome, Parkinson's disease, Long COVID syndrome, drug treatment regimens with IFN-alpha or vitamin A analogues, substance abuse symptoms, and diseases associated with innate inflammatory conditions.

20. The method according to claim 19, wherein the presence or absence of at least one of the following markers are determined in the purified plasma extracellular vesicles: ADAM10, ADAM17, all matrix metalloproteinases, HA, Chitinases, GRASP55, PDL-1, FasL and Hck.

21. A method of treatment or prevention of a condition selected from the group consisting of: major depressive disorder, mood disorders, anxiety-related disorders and depression associated with diseases or drug treatments, HIV associated neurocognitive disorders (HAND), psoriasis, chronic fatigue syndrome, Parkinson's disease, Long COVID syndrome, drug treatment regimens with IFN-alpha or vitamin A analogues, and substance abuse symptoms, the method comprising a step of administering to a patient having said condition a pharmaceutically effective amount of compound having the structure of Formula; 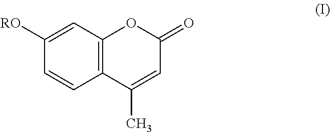
wherein R is H, a phosphate, ester, monosaccharide, or disaccharide; or a salt thereof.

22. The method of treatment according to claim 21, further comprising a step of monitoring the effect of the treatment by analyzing the content of low molecular weight HA in plasma extracellular vesicles in a blood or plasma sample of the patient.

23. The method of treatment according to claim 21, wherein the symptom(s) of the treated Long COVID syndrome is/are depression, anxiety, fatigue, and/or cognitive impairments.

24. A method to modify the presence of hyaluronic acid (HA), and/or cleavage products of HA i-on the surface of extracellular vesicles (EV) or vesicular structures prepared for therapeutic use, via stable or transient transfection of an expression plasmid to an eukaryotic cell producing said vesicles or, alternatively, synthesizing said vesicles in vitro for the delivery of molecular cargo for use in targeting immune cells or the brain for therapeutic, preventive and/or age-reversing effects.

25. (canceled)

26. The method of treatment according to claim 21, wherein R is H and the compound is hymecromone, or a salt thereof.

27. The method of treatment according to claim 21, wherein said condition is major depressive disorder.

28. The method of treatment according to claim 21, wherein said condition is a mood disorder.

29. The method of treatment according to claim 21, wherein said condition is an anxiety-related disorder.

30. The method of treatment according to claim 21, wherein said depression is associated with Alzheimer's disease, psoriasis, chronic fatigue syndrome or Parkinson's disease.

31. The method of treatment according to claim 30, wherein said depression is associated with Alzheimer's disease.

32. The method of treatment according to claim 30, wherein said depression is associated with Parkinson's disease.

33. The method of treatment according to claim 21, wherein said condition is HIV-infection-associated HAND Syndrome.

34. The method of treatment according to claim 21, wherein said condition is Long COVID syndrome.

35. The method of treatment according to claim 21, wherein said condition is a hangover caused by alcohol consumption.

36. The method of treatment according to claim 21, wherein said condition is a depression associated with a drug treatment regimen with IFN-alpha or vitamin A analogues.
